(19) United States
(12) Patent Application Publication
(10) Pub. No.: US 2026/0347760 A1
(43) Pub. Date: Nov. 5, 2026

(54) BACILLUS SUBTILIS STRAIN WITH POSITIVE INFLUENCE ON NEURODEGENERATIVE DISEASES SUCH AS ALZHEIMER'S DISEASE

(71) Applicant: Evonik Operations GmbH, Essen (DE)

(72) Inventors: Juliane PETER, Haltern am See (DE); Stefan PELZER, Gütersloh (DE); Heike TOM DIECK, Friedrichsdorf (DE); Michelle DARGATZ, Bielefeld (DE); Lisa GÜTTLER, Griesheim (DE); Andreas BREMGES, Bielefeld (DE)

(73) Assignee: Evonik Operations GmbH, Essen (DE)

(21) Appl. No.: 19/117,564

(22) PCT Filed: Oct. 4, 2023
(86) PCT No.: PCT/EP2023/077453

§ 371 Date: Apr. 1, 2025

(30) Foreign Application Priority Data
Oct. 7, 2022 (EP) 22200332.9

(51) Int. Cl. C12N 1/205; A61K 35/00; A61K 35/742; A61P 25/28; C12R 1/125

(52) U.S. Cl. CPC C12N 1/205; A61K 35/742; A61P 25/28; A61K 2035/115; C12R 2001/125

(57) ABSTRACT

Summary

The present invention relates to a new B. subtilis strain with positive influence on Alzheimer's disease.

The present invention relates to a new B. subtilis strain with positive influence on Alzheimer's disease, alone and in combination with a booster substance and its use as probiotic. The invention further relates to use the inventive strain as food supplement and as pharmaceutical product.

There are several risk factors which have been identified for neurodegenerative diseases such as obesity, cardiovascular impairment, and diabetes. However, when getting older the risk of cognitive decline or developing a neurodegenerative disease like Alzheimer's disease is increasing exponentially. With an aging population this is leading to a significant number of people affected by cognitive diseases. This increase is a challenge for the patients, their families, and the health care system. It is known that neurodegenerative diseases such as Alzheimer's disease and dementia can be found in people already 20 years before symptoms start. Therefore, a focus is on the reduction of risk factors developing neurodegenerative diseases and by that have the potential to prevent and/or cure such diseases.

New developments have found a connection between the gut and the brain that can be influenced by the gut microflora. The current invention concerns a new B. subtilis strain that showed effects in postponing neurodegenerative decline and the Alzheimer's status in different types of Caenorhabditis elegans.

In the state of art it is described that B. subtilis (NCIB3610 (DSM 10) and JH642) fed to C. elegans types N2, CF1038 and PS3551 showed different life prolonging effects compared to C. elegans fed with typical E. coli OP50 bacteria due to biofilm formation and the production of nitric oxide and the quorum-sensing pentapeptide CSF. When all of these genes (biofilm formation, NO, CSF) were deactivated the lifespan of C. elegans was decreased (Donato, V et al. Bacillus subtilis biofilm extends Caenorhabditis elegans longevity through downregulation of the insulin-like signalling pathway. Nat. Commun. 8, 14332 doi: 10.1038/ncomms14332 (2017)). Another publication researched on the Anti-Alzheimer's effects of B. subtilis NCIB3610 (DSM 10) on certain C. elegans types due to biofilm formation and the production of the quorum-sensing pentapeptide CSF. C. elegans mutants CL2120 and GMC101 that express A-beta-proteins in the muscle cells of the worms fed with the B. subtilis showed delayed the neuronal deterioration could be delayed, slower paralysis and performed better in behavioural tests. Equal effects could be observed in the C. elegans mutant CL2355 with pan-neuronal expressed A-beta-proteins (Cogliati, S et al. Bacillus Subtilis Delays Neurodegeneration and Behavioral Impairment in the Alzheimer's Disease Model Caenorhabditis Elegans. Journal of Alzheimer's disease: JAD 73 (3): 1-18 doi: 10.3233/JAD-190837 (2019)). Furthermore, B. subtilis strains NCIB3610 (DSM 10), 168 (DSM 23778) and JH642 showed a protective effect against a-synuclein aggregation in the C. elegans mutant NL5901, expressing human α-synuclein (Goya, M et al. Probiotic Bacillus subtilis Protects against α-Synuclein Aggregation in C. elegans. Cell Rep, 30 (2), 367-380.e367. https://doi.org/10.1016/j.celrep.2019.12.078 (2020)).

The biofilm producing capability of the described B. subtilis NCIB3610 (DSM 10) was tested in the biofilm promoting MSgg medium as well as in a liquid standard medium NGM only. To ensure the biofilm formation and therefore the bacterial colonization of a probiotic ingredient it needed to be tested under colon similar conditions.

Therefore, it was an objective of the present invention to provide a probiotic strain, which is able to reduce the risk to develop neurodegenerative diseases by microbiome modulation under colon similar conditions.

The use of B. subtilis strains as probiotic ingredient in the feed industry has been disclosed before in the state of the art. The function of probiotics (also called “direct-fed microbials” or “DFM”) is to influence the gut microflora in a positive way by supporting the growth of beneficial bacteria and/or the suppression of the growth of pathogenic bacteria.

Many neurodegenerative diseases do not have one clear pathway in the body but are multifactorial disease. The invention is targeting multiple pathways and shows a positive influence and an in-situ production of several metabolites.

Surprisingly, the new B. subtilis strain DSM 34350 also showed efficient biofilm formation in human simulated-colonic-environment medium (SCEM), whereas the B. subtilis NCIB3610 (DSM 10), 168 (DSM 23778) and JH642 described by the state of the art had a similar growth rate but were not able to build a biofilm under human colon conditions. Also, the new strain showed significantly higher fibrinolytic enzyme activity under human colon conditions compared to the described strains. Fibrinolytic enzymes such as Nattokinase are able to break down protein plaques such as amyloid fibrils that accumulate in the brain and other organs of Alzheimer patients. Therefore, a higher fibrinolytic activity is thought to be beneficial concerning the prevention of amyloid plaque formation.

Further tests in different types of C. elegans confirmed significant differences. It could be shown that paralysis in C. elegans mutant GMC101 could be significantly postponed, that means the lifespan could be prolonged compared to worms eating E. coli OP50 or B. subtilis NCIB3610 (DSM 10).

New insights could be gained in another experiment where B. subtilis DSM 34350 was fed to C. elegans to examine aging related neurodegeneration. Here also significant life prolonging effects could be observed.

Bacillus subtilis DSM 34350 has been identified by targeted screening of naturally occurring isolates. It has been deposited with the Leibniz-Institut DSMZ Deutsche Sammlung von Mikroorganismen and Zellkulturen GmbH, Inhoffenstr. 7B, 38124 Braunschweig, Germany on Aug. 16, 2022 under the provisions of the Budapest Treaty on the International Recognition of the Deposit of Microorganisms for the Purpose of Patent Procedure under the Accession Number as mentioned before in the name of Evonik Operations GmbH.

Therefore, a first subject of the current invention is the B. subtilis strain as deposited under DSM 34350 at the DSMZ; or a preparation thereof.

The Bacillus subtilis strain as deposited under DSM 34350 at the DSMZ exhibits the following characterizing sequences:
a) a 16S rDNA sequence with a sequence identity of at least 99.5%, preferably at least 99.8%, above all 100%, to the polynucleotide sequence according to SEQ ID NO: 1;
b) a yqfD sequence with a sequence identity of at least 99.5%, preferably at least 99.8%, above all 100%, to the polynucleotide sequence according to SEQ ID NO: 2;
c) a gyrB sequence with a sequence identity of at least 99.5%, preferably at least 99.8%, above all 100%, to the polynucleotide sequence according to SEQ ID NO: 3;
d) an rpoB sequence with a sequence identity of at least 99.5%, preferably at least 99.8%, above all 100%, to the polynucleotide sequence according to SEQ ID NO: 4;
e) a groEL sequence with a sequence identity of at least 99.5%, preferably at least 99.8%, above all 100%, to the polynucleotide sequence according to SEQ ID NO: 5.

Thus, a further subject of the current invention is a Bacillus subtilis strain, in particular a B. subtilis strain with the characteristics as mentioned before, or a preparation thereof, wherein the B. subtilis strain exhibits at least one, preferably all, of the following characteristics:
a) a 16S rDNA sequence with a sequence identity of at least 99.5%, preferably at least 99.8%, above all 100%, to the polynucleotide sequence according to SEQ ID NO: 1;
b) a yqfD sequence with a sequence identity of at least 99.5%, preferably at least 99.8%, above all 100%, to the polynucleotide sequence according to SEQ ID NO: 2;
c) a gyrB sequence with a sequence identity of at least 99.5%, preferably at least 99.8%, above all 100%, to the polynucleotide sequence according to SEQ ID NO: 3;
d) an rpoB sequence with a sequence identity of at least 99.5%, preferably at least 99.8%, above all 100%, to the polynucleotide sequence according to SEQ ID NO: 4;
e) a groEL sequence with a sequence identity of at least 99.5%, preferably at least 99.8%, above all 100%, to the polynucleotide sequence according to SEQ ID NO: 5.

Thus, a particular subject of the current invention is also a Bacillus subtilis strain, exhibiting the following characteristics:
a) a 16S rDNA sequence according to SEQ ID NO: 1;
b) a yqfD sequence according to SEQ ID NO: 2;
c) a gyrB sequence according to SEQ ID NO: 3.
d) an rpoB sequence according to SEQ ID NO: 4;
e) a groEL sequence according to SEQ ID NO: 5.

The strain of the current invention are preferably characterized by at least one, more preferably by all, of the following further features:

The strain is preferably able to grow under anaerobic conditions. Further, it is preferably able to grow under human colon conditions.

The B. subtilis strain is characterized by being able to build a biofilm under colon conditions after 24 h at 37 °C, preferably with a biofilm intensity of at least 0.1, determined by measurement of absorption at 565 nm.

It is preferred, when the biofilm is built in SCEM medium or in TSB medium at pH 7.

In particular, the B. subtilis strain is characterized by having a fibrinolytic activity under colon conditions, preferably a nattokinase activity, preferably determined by an average halo diameter of at least 20 mm produced on fibrin agar plates after 24 h.

Preferably, the B. subtilis strain is characterized by an ability to produce short chain fatty acids under colon conditions, preferably acetate, butyrate and lactate, preferably more than 0.1 g/l or more than 0.2 g/l after 26 h.

Without wishing to be bound by any theory, it is thought that the Bacillus subtilis strain according to the current invention enhance human health, in particular gut health or mental health, by a multifaceted mode of action, including fibrinolytic activity and the production of short chain fatty acids. As many neurodegenerative diseases do not have one clear pathway in the body, but are multifactorial disease, multiple pathways are targeting with the present invention and show a positive influence and an in-situ production of several metabolites.

In a preferred embodiment of the current invention, the strain and preparations of the present invention are administered orally to animals or human beings.

Thus, a further subject of the current invention are compositions, such as feedstuffs, foodstuffs, drinking and rearing water as well as therapeutic compositions, containing a B. subtilis strain and/or a preparation thereof according to the current invention.

A further subject of the current invention is also the use of a B. subtilis strain and/or a preparation of the current invention as a probiotic ingredient (DFM) in feed or food products.

Preferred foodstuffs according to the invention are dairy products, in particular yoghurt, cheese, milk, butter and quark.

The cells of the strain of the current invention may be present, in particular in the compositions of the current invention, as spores (which are dormant), as vegetative cells (which are growing), as transition state cells (which are transitioning from growth phase to sporulation phase) or as a combination of at least two, in particular all of these types of cells. In a preferred embodiment, the composition of the current invention comprises mainly or only spores.

In addition, or as alternative the cells of the strains may also be used in non-living, inactivated form, as also the non-living cells are expected to still have a probiotic effect. Ways to inactivate the cells are known to those skilled in the art.

The methods and uses of the strain and the preparations of the current invention can be therapeutic or non-therapeutic.

A further subject of the present invention is a food- or feedstuff composition containing the B. subtilis strain according to the present invention or a preparation thereof and at least one further feed or food ingredient selected from proteins, carbohydrates, fats, further probiotics, prebiotics, enzymes, vitamins, immune modulators, milk replacers, minerals, amino acids, coccidiostats, acid-based products, medicines, and combinations thereof, preferably manganese or thiamin.

A further subject of the current invention is therefore also a pharmaceutical composition comprising the strain and/or preparation of the current invention as mentioned before and a pharmaceutically acceptable carrier.

A particular subject of the current invention is also a method of enhancing the health of human beings and/or of improving the general physical condition of human beings and/or of increasing the disease resistance of human beings and/or of increasing the immune response of human beings and/or of establishing or maintaining a healthy gut microflora in human beings, wherein the strain and/or preparations of the current invention are administered to human beings.

A further subject of the current invention is therefore also the use of strain and/or preparations of the current invention for enhancing the health of human beings and/or for improving the general physical condition of human beings and/or for increasing the disease resistance of human beings and/or for increasing the immune response of human beings and/or for establishing or maintaining a healthy gut microflora in human beings, wherein the strains and/or preparations of the current are administered to human beings.

Another aspect of the invention is directed to a pharmaceutical or non-pharmaceutical composition which further comprises a targeted-release formulation for delayed release or enteric or colonic release. A targeted-release formulation according to the present invention is a formulation which ensures the delivery of the component of the preparation according to the present invention to a specific target in the body. A preferred formulation of such preparations promotes enteral or colonic delivery in the lower small intestine or in the large intestine. The targeted-release formulation can be obtained by adding enteric polymers to the matrix of the dosage form, or by adding a coating to the dosage form, preferably an enteric coating.

According to the present invention, a colon-specific delivery system is a delivery system, which targets the substance or drug directly to the colon. The advantage of a colon-specific delivery system is the local action, in case of disorders like ulcerative colitis, Crohn's disease, irritable bowel syndrome, and carcinomas. Targeted drug delivery to the colon in these cases ensures direct treatment at the site with lower dosing and fewer systemic side effects. In addition to local therapy colon can also be utilized as the portal entry of the drugs into systemic circulation for example molecules that are degraded/poorly absorbed in upper gut such as proteins and peptides may be better absorbed from the more benign environment of the colon. Colon-specific drug delivery is considered beneficial in the treatment of colon-related diseases and the oral delivery of protein and peptide drugs. Generally, each colon-specific drug delivery system has been designed based on one of the following mechanisms with varying degrees of success; 1. Coating with pH dependent polymers, 2. Coating with pH independent biodegradable polymers and 3. Delivery systems based on the metabolic activity of colonic bacteria.

An enteric coating is a barrier applied on oral medication that prevents its dissolution or disintegration in the gastric environment. Most enteric coatings work by presenting a surface that is stable at the intensely acidic pH found in the stomach but breaks down rapidly at a higher pH (alkaline pH). For example, they will not dissolve in the gastric acids of the stomach (pH ~3), but they will start to dissolve in the environment present in the distal small intestine (pH range proximal to distal small intestine is ~5.6 to 7.4). Colon targeted (drug) delivery systems are designed to selectively release a drug in response to the colonic environment without premature drug release in the upper GI tract.

The colon-specific delivery system can comprise a pH-dependent drug delivery system, since the colon exhibits a relatively higher pH than the upper GI tract. Accordingly, a colon-targeted delivery system is designed by using pH-dependent polymers such as cellulose acetate phthalates (CAP), hydroxypropyl methyl-cellulose phthalate (HPMCP) 50 and 55, copolymers of methacrylic acid and methyl methacrylate (e.g., Eudragit® S 100, Eudragit® L, Eudragit® FS, and Eudragit® P4135 F).

Therefore, in an advantageous configuration, the colon-specific delivery system comprises a coating comprising at least one pH dependent polymer or biodegradable polymer, preferably selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein.

As a coating it is preferred to use a polymer polymerized from 10 to 30% by weight methyl methacrylate, 50 to 70% by weight methyl acrylate and 5 to 15% by weight methacrylic acid.

The polymer dispersion as disclosed may preferably comprise 15 to 50% by weight of a polymer polymerized from 20 to 30% by weight methyl methacrylate, 60 to 70% by weight methyl acrylate and 8 to 12% by weight methacrylic acid. Most preferred the polymer is polymerized from 25% by weight methyl methacrylate, 65% by weight methyl acrylate and 10% by weight methacrylic acid.

A 30% by weight aqueous dispersion of a polymer polymerized from 25% by weight methyl methacrylate, 65% by weight methyl acrylate and 10% by weight methacrylic acid corresponds to the commercial product EUDRAGUARD® biotic.

The percentages of the monomers add up to 100%. The functional polymer is applied in amounts of 2-30 mg/cm², preferably 5-20 mg/cm².

Another preferred subject in this context is the use of the pharmaceutical compositions as described before as a medicament. The present invention provides a therapeutic composition for use in the prevention or treatment of Alzheimer's disease.

The compositions of the present invention, in particular the feed, food and pharmaceutical compositions as well as the drinking or rearing water, preferably comprise the strains of the current invention and are administered to animals at a rate of about 1×10³ to about 2×10¹² CFU/g feed or ml water, in particular in a rate of about 1×10³ or about 1×10⁴ or about 1×10⁵ or about 1×10⁶ or about 1×10⁷ or about 1×10⁸ or about 1×10⁹ or about 1×10¹⁰ or about 1×10¹¹ or about 1×10¹² CFU/g feed or ml water, preferably in an amount of about 1×10⁴ to about 1×10¹⁰ CFU/g feed or ml water, more preferably in an amount of 1×10⁴ to 1×10⁷ CFU/g feed or ml water.

Correspondingly, preferred amounts of the strains and/or preparations of the current invention in the feed, food and water compositions of the current invention range preferably from 0.1 wt.-% to 10 wt.-%, more preferably from 0.2 wt.-% to 5 wt.-%, in particular from 0.3 wt.-% to 3 wt.-%.

The food- or feedstuff composition according to the present invention does also include dietary supplements in the form of a pill, capsule, tablet or liquid.

Moreover, the present invention is also related to a method of improving the health status, in particular the gut health status of a subject in need, such as a human being or an animal, comprising administering to the human being or animal a B. subtilis strain or a preparation according to the present invention.

The strain and preparations of the present invention can be obtained by culturing the strains of the current invention according to methods well known in the art, including by using the media and other methods as described for example in U.S. Pat. No. 6,060,051, EP0287699 or US2014/0010792. Conventional large-scale microbial culture processes include submerged fermentation, solid state fermentation, or liquid surface culture. Towards the end of fermentation, as nutrients are depleted, the cells of the strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the Bacillus subtilis cells and to promote sporulation. The bacterial cells, spores and metabolites in culture media resulting from fermentation may be used directly or concentrated by conventional industrial methods, such as centrifugation, tangential-flow filtration, depth filtration, and evaporation. The concentrated fermentation broth may be washed, for example via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, as described above, may also be added post-drying.

Preparations of the strain of the current invention may be cell-free preparations or preparations containing cell debris or preparations containing a mixture of intact cells and cell debris.

Cell-free preparations of the strain of the current invention can be obtained for example by centrifugation and/or filtration of fermentation broth. Depending on the technique used, these cell-free preparations may not be completely devoid of cells, but may still comprise a smaller amount of cells. As the cells secret compounds like metabolites, enzymes and/or peptides into the surrounding medium, the supernatant of the cells comprises a mixture of such compounds, in particular metabolites, enzymes and/or peptides, as secreted by the cells. Thus, in a preferred embodiment of the invention, the preparation of the strains is a supernatant of the fermentation broth.

Compositions comprising cell debris of the strains may be obtained by rupturing the cells applying techniques as known to those of skill in the art, for example by mechanical means or by applying high pressure. Depending on the degree of force applied, a composition comprising only ruptured cells or a composition comprising a mixture of cell debris and intact cells is obtained. Homogenization of the cells may be realized for example by utilizing a French cell press, sonicator, homogenizer, microfluidizer, ball mill, rod mill, pebble mill, bead mill, high pressure grinding roll, vertical shaft impactor, industrial blender, high shear mixer, paddle mixer, and/or polytron homogenizer. Suitable alternatives are enzymatic and/or chemical treatment of the cells.

Cell-free preparations of the current invention comprise also preparations which are obtained by first rupturing the cells by applying techniques as mentioned before and subsequently removing the cell debris and the remaining intact cells. Removing of the cell debris and remaining intact cells can be carried out in particular by centrifugation and/or filtration.

The preparations of the strains of the current invention may comprise as active compounds at least one metabolite, preferably a mixture of metabolites and/or at least one enzyme selected from proteases, in particular nattokinase, subtilisin, xylanases and/or cellulases, and/or at least one peptide, and/or combinations thereof.

A preparation containing an effective mixture of metabolites as contained in the strain of the current invention and/or as contained in the cell preparations as mentioned before, can be obtained for example according to the methods set forth in U.S. Pat. No. 6,060,051. In particular the preparation can be obtained by precipitating the metabolites as contained in the preparations mentioned before by using organic solvents like ethyl acetate and subsequent redissolving of the precipitated metabolites in an appropriate solvent. The metabolites may subsequently be purified by size exclusion filtration that groups metabolites into different fractions based on molecular weight cut-off.

Preferably according to the invention always an effective amount of the strain and/or preparations of the current invention is used in the embodiments of the current invention. The term “effective amount” refers to an amount which effects at least one beneficial effect to an animal and/or to the environment, in particular with respect to the features as already mentioned before, in comparison to an animal that has not been administered the strains and/or preparations and/or compositions of the current invention, but besides that has been administered the same diet (including feed and other compounds).

In case of therapeutic applications preferably a therapeutic amount of the strain and/or preparations of the current invention is used. The term “therapeutic amount” refers to an amount sufficient to ameliorate, reverse or prevent a disease state in a human being or an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance human or animal health, in particular gut health, at various doses.

### EXAMPLES

### Methods

### 1. Biofilm Assay

Substances produced by Bacillus subtilis DSM10 under biofilm formation were associated with anti-aging effects and shown to be relevant for live prolongation of Caenorhabditis elegans wild type strains and disease onset delay in C. elegans Alzheimer mutants (Donato, Ayala et al. 2017, Cogliati, Clementi et al. 2020).

The testing strain B. subtilis DSM 34350 and the reference strain B. subtilis DSM10 were revitalized by spreading 50 μl of glycerol cryo stocks on tryptic soy agar (TSA) and incubation for 24 h at 37 °C. Cell material from the plate was used to inoculate a preculture of 10 ml tryptic soy broth (TSB) in 100 ml Erlenmeyer flasks. The flasks were incubated for 16 h at 200 rpm and 37 °C.

The biofilm assay was conducted under colon conditions in human simulated-colonic-environment medium (SCEM) containing 6.25 g/l Bacto tryptone (BD), 2.6 g/l D-Glucose, 0.88 g/l NaCl, 2.7 g/l KHCO₃, 0.43 g/l KH₂PO₄, 1.7 g/l NaHCO₃ and 4 g/l Bile salts no. 3 at pH 7 and in TSB as a control. The preculture of the testing strain was used to inoculate 200 μl SCEM medium in a 96-well plate to an optical density OD600 of 0.2 in six replicates. The plate was incubated without shaking at 37 °C allowing biofilm formation. After 24 h the absorption at 600 nm was measured from each well at a Tecan Spark device to determine the general ability to grow in the respective medium. Then, cultures were removed from the wells by inverting the plate and shaking out the contents in a waste container. The wells were washed twice by completely filling each well with sterile water and inverting the plate and shaking out the contents. Biofilms that remain attached to the well were stained by adding 250 μl of 0.1% crystal violet solution and incubation for 30 min at room temperature (RT). Afterwards, all wells were washed three or four time with sterile water until no staining could be washed out anymore. The plate was allowed to dry over night at room temperature before the stained biofilm was resolved by adding 225 μl of 99.9% ethanol and incubation for 15 min at RT. The absorption of the stained biofilm was measured at 595 nm in a Tecan Spark device (100 flashes, 100 ms rest time). In case the absorption was above the linear range of the device, samples were diluted in ethanol and measured again.

### 2. Fibrinolysis Assay

The fibrinolysis assay was applied to evaluate the fibrinolytic activity by enzymes such as the Nattokinase of the test strain under human colon conditions. Fibrinolytic activities are required for breaking down protein plugs such as the otherwise insoluble misfolded amyloid fibrils that accumulate in the brain and other organs of Alzheimer patients (Hsu, Lee et al. 2009, Fadl, Ahmed et al. 2013).

Solutions used were a fibrinogen stock (0.6 g/100 ml in 50 mM pH 7.4 sodium phosphate buffer), a thrombin Stock (10 μg/ml in 50 mM pH 7.4 sodium phosphate buffer), agarose solution (2% agarose in 50 mM pH 7.4 sodium phosphate buffer) and a plasmin stock (10 μg/ml in 100 mM sodium phosphate, 25% glycerol, pH 7.3). Fibrin agar plates were freshly prepared prior to each testing by mixing 5 ml fibrinogen Stock, 5 ml agarose solution and 0.1 ml thrombin stock in a Petri dish and allowing it to polymerize for 1 h at RT prior to usage.

The testing strain B. subtilis DSM 34350 and the reference strain B. subtilis DSM10 were revitalized by spreading 50 μl of glycerol cryo stocks on TSA and incubation for 24 h at 37 °C. Cell material from the plate was used to inoculate a preculture of 10 ml SCEM and in 10 ml in TSB as a control in 100 ml Erlenmeyer flasks. The flasks were incubated for 16 h at 200 rpm and 37 °C. The cultures were diluted to an OD 600 of 2 in SCEM and 50 μl of the diluted culture were applied into a 9 mm diameter well cut into the fibrin agar. Pure SCEM was applied as a negative control, 50 μl of plasmin stock as a positive control. The plates were incubated for 24 h and the diameter of the transparent halo was measured, which is proportional to the fibrinolytic activity of the test strain.

### 3. Short Chain Fatty Acid (SCFA) Production

The production of SCFA by the gut microbiota is associated with diverse beneficial effects on the host and was also described to modulate the metabolic fitness of the brain (Erny, Dokalis et al. 2021). Decreased amounts of SCFA are correlated with the induction of inflammatory processes in the body, affecting the permeability of the blood-brain-barrier and enabling neuroinflammation (Eicher and Mohajeri 2022). Mainly butyrate and acetate are described to exert positive effect on the brain health, however lactate produced by the B. subtilis strain can serve as a substrate for cross-feeding commensal bacteria to produce butyrate in the human gut.

The testing strain DSM 34350 was revitalized by spreading 50 μl of glycerol cryo stocks on TSA and incubation for 24 h at 37 °C. Cell material from the plate was used to inoculate a preculture of 10 ml TSB in 100 ml Erlenmeyer flasks. The flasks were incubated for 16 h at 200 rpm and 37 °C.

Short chain fatty acid (SCFA) production of the strain DSM 34350 was tested under human colon conditions in SCEM. The preculture of the testing strain was used to inoculate 1000 μl SCEM medium in a 48-well plate to an optical density OD600 of 0.2 in three replicates. The plate was incubated for 26 h at 37 °C and 400 rpm and anaerobic conditions. The SCFA acetate and lactate were quantified via high performance liquid chromatography.

4. Analysis of Anti-Alzheimer Effects in C. elegans

The in vivo anti-Alzheimer effect of Bacillus subtilis DSM10 and DSM 34350 was tested in the transgenic C. elegans strain GMC101. This mutant expresses the of full-length human A-beta-1-42 peptide, which builds the extracellular insoluble amyloid plaques found in human Alzheimer's brains. The peptide is produced in the body wall muscle cells of the worm and under control of a temperature sensitive promoter. Shifting larval stage 4 or young adult animals from 20 °C to 25 °C causes expression of the peptide in the muscle cells and leads to rapid paralysis (inability to move) of the worms.

For testing the strains, both Bacillus subtilis strains and the standard non-probiotic C. elegans feeding strain Escherichia coli OP50 were cultivated at 37 °C during 24 h (200 rpm) in TSB medium and harvested by spinning down at 3000 rpm for 15 min. For each probiotic, the supernatant was carefully removed, and the resulting pellet was resuspended in S-medium buffer (5.85 g/l NaCl, 1 g/l K₂HPO₄, 6 g KH₂PO₄, H₂O, 1 m/L cholesterol (5 mg/ml in ethanol), 3 ml/l 1M of CaCl₂), 3 ml/l 1 M MgSO₄, 10 ml/l 1 M potassium citrate, 10 ml/l of Trace metals solution [1.86 g disodium EDTA, 0.69 g FeSO₄×7 H₂O, 0.2 g MnCl₂×4 H₂O, 0.29 g ZnSO₄×7 H₂O, 0.025 g CuSO₄×5 H₂O, H₂O to 1 L], and 0.6 ml/l TWEEN20) to reach an OD 600. 50 μl of the resuspended cultures were seeded on Nematode Growth Medium (NMG) Agar containing 3 g/l NaCl, 2.5 g/l peptone, 20 g/l agar, 1 ml/l of cholesterol (5 mg/mL in ethanol), 1 ml/l of 1 M CaCl₂, 1 ml/l 1 M MgSO₄, and 25 ml/l of 1 M (pH 6.0) KPO₄ dried overnight at room temperature.

Worms were maintained on NGM agar plates containing the bacterial strain of interest from the L1 larval stage to the L4 larval stage at 20 °C. The worms were then transferred to probiotic or feeding strain plates and incubated at 25 °C to induce amyloid expression. The worm populations were transferred to freshly seeded plates containing the strain of interest every second day. Per strain, five plates (n=5) with 10 worms per plate were tested.

Worm populations were observed under a stereomicroscope once a day. Before each motility monitoring, the plates containing worms were tapped on the bench three times to ensure a homogeneous stimulation among the population. Following this first stimulation, three parameters were recorded successively:
the spontaneous movement with no further stimulation (worm's capacity to move is observed over a period of 30 s). If not observed;
the movement of the head or the tail after stimulations (one gentle touch of the head and the tail with a worm's pick). If not observed;
the paralysis (no response after three additional stimulations)

Motility endpoints obtained every day for each plate containing 10 worms were employed to compute the percentage of worms showing (1) spontaneous movements; (2) movements of the head/tail; (3) paralyzed. The percentages obtained for each condition were compared to the control (worms fed with OP50 bacteria). The time point DO correspond to the time point just before the transfer of the population at 25 °C: the whole population was considered 100% mobile at this time point. In all experiments, statistical analysis was conducted by using two-way analysis of variance (ANOVA-2) followed by Bonferroni's multiple comparisons tests. All P values <0.05 were considered to be significant (*p<0.5; **p<0.01; ***p<0.001; ****p<0.0001 are symbols representing the statistical value following the bonferroni's mutliple test/$ p<0.5; $$ p<0.01; $$$ p<0.001; $$$$ p<0.0001 are symbols representing the statistical value following the ANOVA-2, which explain the variations between conditions). GraphPad Prism 5 was used for all statistical analyses.

5. Identification of Compounds with Biofilm-Boosting Effect

As biofilm formation capability of Bacillus subtilis was associated with anti-aging effects, supplemental ingredients with a potential biofilm enhancing effect in strain DSM 34350 were tested.

The testing strain B. subtilis DSM 34350 was revitalized by spreading 50 μl of glycerol cryo stocks on tryptic soy agar (TSA) and incubation for 24 h at 37 °C. Cell material from the plate was used to inoculate a preculture of 10 ml tryptic soy broth (TSB) in 100 ml Erlenmeyer flasks. The flasks were incubated for 16 h at 200 rpm and 37 °C.

For the biofilm assay was conducted with and without the testing substances manganese, thiamine, and a combination of phenylalanine and tryptophane under colon conditions in SCEM. For testing the substances, 0.015-0.04 mg/ml manganese in forms of manganous sulfate tetrahydrate, 0.68 μg/ml thiamine in forms of Thiamine×HCl and a combination of each 0.05 mg/ml phenylalanine and tryptophane were added individually to the SCEM medium and the biofilm formation capability was considered for each additive in comparison to the SCEM control.

The preculture of the testing strain was used to inoculate 200 μl of each medium variation in a 96-well plate to an optical density OD600 of 0.2 in six replicates. The plate was incubated without shaking at 37 °C allowing biofilm formation. After 24 h the absorption at 600 nm was measured from each well at a Tecan Spark device to determine the general ability to grow in the respective medium. Then, cultures were removed from the wells by inverting the plate and shaking out the contents in a waste container. The wells were washed twice by completely filling each well with sterile water and inverting the plate and shaking out the contents. Biofilms that remain attached to the well were stained by adding 250 μl of 0.1% crystal violet solution and incubation for 30 min at room temperature (RT). Afterwards, all wells were washed three or four times with sterile water until no staining could be washed out anymore. The plate was allowed to dry over night at room temperature before the stained biofilm was resolved by adding 225 μl of 99.9% ethanol and incubation for 15 min at RT. The absorption of the stained biofilm was measured at 595 nm in a Tecan Spark device (100 flashes, 100 ms rest time). In case the absorption was above the linear range of the device, samples were diluted in ethanol and measured again.

6. Comparison with Other Strains in the Context of the Treatment of Neurodegenerative Diseases

To compare the described strain DSM 34350 to other state of the art strains described in the context of the treatment of neurodegenerative diseases, following strains tested in the biofilm formation and fibrinolysis assay described above (1. and 2.) under colon conditions (SCEM medium):

TABLE 1
Bacillus subtilis state of the art strains (DSM10 and
JH642) selected for comparison in fibrinolysis and biofilm
formation assay with DSM 34350, complemented by additional
benchmark strains (DSM1090, SMY, PY79 and DSM23778) with
high genome-wide Average Nucleotide Identity (gANI).

| Strain | Context |
|---|---|
| DSM10 | Cogliati, S. V. et al. (2020) |
| JH642 | Goya, M. E., et al. (2020). |
| DSM1090 | >99.98% gANI to JH642 |
| | >99.97% gANI to DSM10 |
| SMY | >99.98% gANI to DSM10 |
| | >99.97% qANI to JH642 |
| PY79 | >99.95 gANI to JH642 |
| | >99.94% gANI to DSM10 |
| DSM23778 | >99.98 gANI to JH642 |
| | >99.99% gANI to DSM10 |

The biofilm assay was conducted in six technical replicates and the fibrinolysis assay in three technical replicates. Statistical analysis was conducted by using one-way analysis of variance.

### Results

### 1. Biofilm Formation Under Colon Conditions

Biofilm formation under colon-similar conditions was tested for both B. subtilis DSM10 and DSM 34350 by applying a crystal violet-based biofilm assay. Both general growth in terms of absorption at 600 nm and biofilm intensity after crystal violet staining were measured in TSB and SCEM medium (Table 2). While both strains showed growth in both media, only DSM 34350 was able to establish a biofilm in SCEM medium. In full medium (TSB), both strains built a biofilm, but DSM 34350 led to a more than sixfold higher biofilm intensity.

TABLE 2
Growth and biofilm formation of B. subtilis DSM 34350 and B. subtilis
DSM10 in TSB and SCEM medium. Values are averages of six replicates.

| | TSB | | SCEM | |
|---|---|---|---|---|
| | Growth | Biofilm intensity | Growth | Biofilm intensity |
| | [Absorption at | [Absorption at | [Absorption at | [Absorption at |
| Strain | 600 nm] | 565 nm] | 600 nm] | 565 nm] |
| DSM 34350 | 0.82 | 1.83 | 0.27 | 1.78 |
| NCIB3610 | 0.36 | 0.27 | 0.30 | 0.02 |
| (DSM10)/DSM10 | | | | |

### 2. Nattokinase Activity Under Colon Conditions

The fibrinolytic activity of enzymes such as nattokinase was tested under colon-similar conditions for both B. subtilis DSM 10 and DSM 34350 by applying plate-based fibrinolysis assay. The average clearance halo after 24 h was measured for both strains grown in TSB as a control and in SCEM and is proportional to the fibrinolytic activity (Table 3). B. subtilis DSM 34350 showed significantly more fibrinolytic activity in both media than DSM10. In SCEM, the activity of B. subtilis DSM 34350 increased in comparison to TSB and resulted in almost 10 mm increased halo size compared to B. subtilis DSM10.

TABLE 3
Nattokinase activity of B. subtilis DSM 34350 and
B. subtilis DSM10 in TSB and SCEM medium as average
halo diameters produced on fibrin agar plates.

| | TSB | SCEM |
|---|---|---|
| | Average halo | Average halo |
| Strain | diameter [mm] | diameter [mm] |
| DSM 34350 | 22.90 | 26.17 |
| NCIB3610 | 17.77 | 16.63 |
| (DSM10)/DSM10 | | |

### 3. Production of Short Chain Fatty Acids (SCFA)

B. subtilis DSM 34350 was cultivated under anaerobic condition in SCEM and the production of the short chain fatty acids acetate and DL-Lactate was quantified after 26 h. The strain was able to produce both SCFAs under human colon-similar conditions, providing potential substrate for cross-feeding other commensal microbes in the human gut in order to generate butyrate (Table 4).

TABLE 4
Production of the short chain fatty acids acetate and DL-Lactate
by B. subtilis DSM 34350 under anaerobic conditions in SCEM

| Strain | Acetate [g/l] | DL-Lactate [g/l] |
|---|---|---|
| DSM 34350 | 0.31 | 0.88 |

4. Analysis of Anti-Alzheimer Effects in C. elegans

The anti-Alzheimer effect of B. subtilis DSM10 and B. subtilis DSM 34350 was evaluated in the transgenic C. elegans strain GMC101, expressing human amyloid-beta in muscle cells, which results in an increasing paralysis and in reduced spontaneous movement over time.

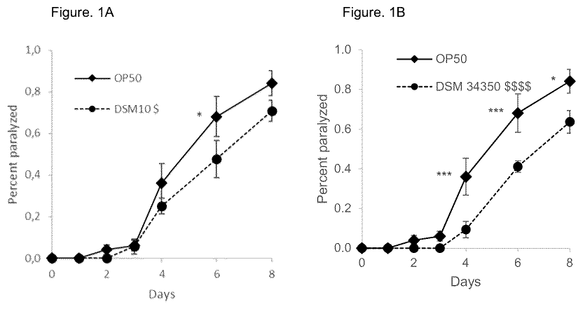

Feeding GMC101 with B. subtilis DSM10 significantly (p<0.05) delayed overall paralysis in comparison to the non-probiotic strain E. coli OP50 (FIG. 1A). For B. subtilis DSM 34350 even higher significant delay of the paralysis proceeding (p<0.0001) was found (FIG. 1B). Regarding the single time points, a significantly decreased percentage of paralyzed worms was found at day 6 for B. subtilis DSM10 and at day 4, 6 and 8 for B. subtilis DSM 34350. After 8 days, less than 65% of the worms paralyzed when fed with B. subtilis DSM 34350, while 84% of the worms fed with E. coli OP50 are paralyzed at the same age.

FIG. 1 shows a paralysis assay results for C. elegans GMC101 maintained at 25 °C fed with E. coli OP50 compared to fed with either A) B. subtilis DSM10 or B) B. subtilis DSM 34350 in comparison over 8 days starting at larval stage L4. Statistical analysis was conducted by using two-way analysis of variance (ANOVA-2) followed by Bonferroni's multiple comparisons tests. All P values <0.05 were considered to be significant (*p<0.5; **p<0.01; ***p<0.001; ****p<0.0001 are symbols representing the statistical value following the Bonferroni's mutliple test/$ p<0.5; $$ p<0.01; $$$ p<0.001; $$$$ p<0.0001 are symbols representing the statistical value following the ANOVA-2, which explain the variations between conditions).

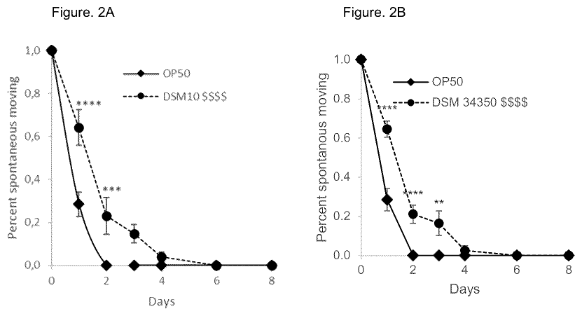

A complete stop of spontaneous movement, which is recorded without any stimulation of the worms, was found for E. coli OP50 fed groups after two days (FIGS. 2A and B). Both B. subtilis DSM10 and B. subtilis DSM 34350 showed significant effects, by delaying the inability for spontaneous movement for two to three days. Complete stop of spontaneous movement was found for both strains only after 6 days. A significant difference for the single time points was found for B. subtilis DSM10 until day 2 and until day 3 for B. subtilis DSM 34350.

FIG. 2 shows spontaneous movement assay results for C. elegans GMC101 maintained at 25 °C fed with E. coli OP50 compared to fed with either B. subtilis DSM10 or B. subtilis DSM 34350 in comparison over 8 days starting at larval stage L4. Statistical analysis was conducted by using two-way analysis of variance (ANOVA-2) followed by Bonferroni's multiple comparisons tests. All P values <0.05 were considered to be significant (*p<0.5; **p<0.01; ***p<0.001; ****p<0.0001 are symbols representing the statistical value following the bonferroni's mutliple test/$ p<0.5; $$ p<0.01; $$$ p<0.001; $$$$ p<0.0001 are symbols representing the statistical value following the ANOVA-2, which explain the variations between conditions).

In order to evaluate a significant difference between B. subtilis DSM10 and B. subtilis DSM 34350, a second ANOVA-2 (DSM 34350 vs DSM10) was performed (Table 5). B. subtilis DSM 34350 significantly improves the worm's motility when compared to B. subtilis DSM10 treatment.

TABLE 5
ANOVA-2 results of comparing B. subtilis
DSM 34350 with B. subtilis DSM10

| Strain | p-value | Variation |
|---|---|---|
| DSM 34350 | * (p < 0.05) | increase |

5. Identification of Compounds with Biofilm-Boosting Effect

As biofilm formation capability of Bacillus subtilis was associated with anti-aging effects, supplemental ingredients with a potential biofilm enhancing effect in strain DSM 34350 were tested. The substances manganese, thiamine, and a combination of phenylalanine and tryptophane were investigated on their effect of DSM 34350 biofilm formation under colon conditions in SCEM.

When comparing the intensity of the biofilm build by DSM 34350, increased values were observed for all tested substances and concentrations in comparison to the control without a substance added (Table 6). For manganese with different concentration, a reverse dose effect was observed: with decreasing manganese added, higher biofilm formation was triggered. 0.05 mg/ml thiamin led to an increase in biofilm formation comparable with the lowest concentration of manganese, while the combination of phenylalanine and tryptophane increased biofilm formation only marginally in comparison to the control.

TABLE 6
Growth and biofilm formation of B. subtilis DSM 34350
in SCEM medium with and without potential boosting
compounds. Values are averages of six replicates.

| | Added compound | | | | | |
|---|---|---|---|---|---|---|
| | | | | | | Phe + |
| | Control | Manganese | | | Thiamin | Trp |
| | Unit | | | | | |
| | — | mg/ml | mg/ml | mg/ml | mg/ml | μg/ml |
| Concentration | — | 0.15 | 0.08 | 0.04 | each | 0.67 |
| | | | | | 0.05 | |
| Biofilm intensity | 0.10 | 0.19 | 0.28 | 0.34 | 0.35 | 0.12 |
| [Absorption | | | | | | |
| at 565 nm] | | | | | | |

In summary, both manganese and thiamin were identified as suitable additives for triggering biofilm formation in DSM 34350, which was associated with anti-aging and anti-Alzheimer effects (Donato, Ayala et al. 2017, Cogliati, Clementi et al. 2020).

6. Comparison with Other Strains in the Context of the Treatment of Neurodegenerative Diseases

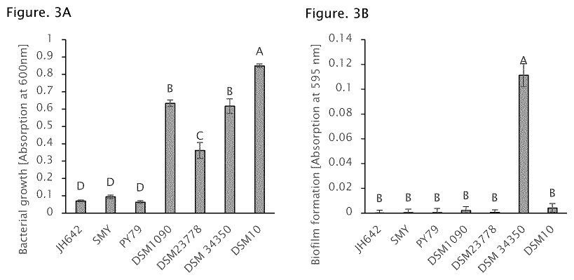

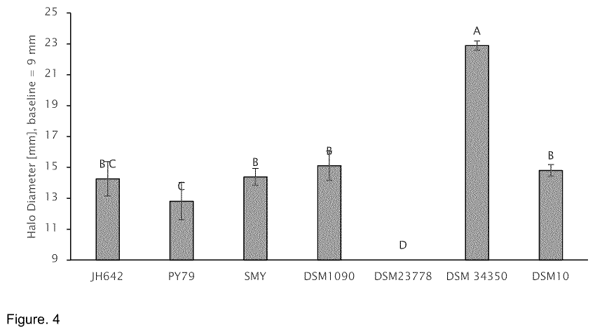

To compare the described strain DSM 34350 to other state of the art strains described in the context of the treatment of neurodegenerative diseases, strains tested in the biofilm formation and fibrinolysis assay under colon conditions (SCEM medium). The biofilm assay was conducted in six technical replicates and the fibrinolysis assay in three technical replicates. The results are depicted in FIGS. 3 and 4. Statistical analysis was conducted by using one-way analysis of variance. Means with the same letter are not significantly different.

While also other strains than the described strain DSM 34350 were able to grow under simulated colon conditions (FIG. 3A), DSM 34350 showed significantly more biofilm formation compared to all other strains (FIG. 3B).

The described strain DSM 34350 showed significantly more fibrinolytic activity under simulated colon conditions compared to all other strains tested (FIG. 4).

### REFERENCES

Cogliati, S., V. Clementi, M. Francisco, C. Crespo, F. Argañaraz and R. Grau (2020). “Bacillus subtilis Delays Neurodegeneration and Behavioral Impairment in the Alzheimer's Disease Model Caenorhabditis Elegans.” J Alzheimers Dis 73 (3): 1035-1052.
Donato, V., F. R. Ayala, S. Cogliati, C. Bauman, J. G. Costa, C. Leñini and R. Grau (2017). “Bacillus subtilis biofilm extends Caenorhabditis elegans longevity through downregulation of the insulin-like signalling pathway.” Nature Communications 8 (1): 14332.
Eicher, T. P. and M. H. Mohajeri (2022). “Overlapping Mechanisms of Action of Brain-Active Bacteria and Bacterial Metabolites in the Pathogenesis of Common Brain Diseases.” Nutrients 14 (13): 2661.
Goya, M. E., et al. (2020). “Probiotic Bacillus subtilis Protects against α-Synuclein Aggregation in C. elegans.” Cell Rep 30 (2): 367-380.e367.
Erny, D., N. Dokalis, C. Mezo, A. Castoldi, O. Mossad, O. Staszewski, M. Frosch, M. Villa, V. Fuchs, A. Mayer, J. Neuber, J. Sosat, S. Tholen, O. Schilling, A. Vlachos, T. Blank, M. Gomez de Agüero, A. J. Macpherson, E. J. Pearce and M. Prinz (2021). “Microbiota-derived acetate enables the metabolic fitness of the brain innate immune system during health and disease.” Cell Metabolism 33 (11): 2260-2276.e2267.
Fadl, N. N., H. H. Ahmed, H. F. Booles and A. H. Sayed (2013). “Serrapeptase and nattokinase intervention for relieving Alzheimer's disease pathophysiology in rat model.” Hum Exp Toxicol 32 (7): 721-735.
Hsu, R. L., K. T. Lee, J. H. Wang, L. Y. Lee and R. P. Chen (2009). “Amyloid-degrading ability of nattokinase from Bacillus subtilis natto.” J Agric Food Chem 57 (2): 503-508.

1. The Bacillus subtilis strain as deposited under DSM 34350 at the DSMZ;
or a preparation thereof.

2. The use of a B. subtilis strain or a preparation thereof according to claim 1 as a probiotic ingredient (DFM) in feed or food products.

3. A food- or feedstuff composition containing a B. subtilis strain or a preparation thereof according to claim 1 and at least one further feed or food ingredient selected from proteins, carbohydrates, fats, further probiotics, prebiotics, enzymes, vitamins, immune modulators, milk replacers, minerals, amino acids, coccidiostats, acid-based products, medicines, and combinations thereof, preferably manganese or thiamin.

4. A pharmaceutical composition containing a B. subtilis strain or a preparation thereof according to claim 1 and a pharmaceutically acceptable carrier.

5. The composition according to claim 3, further comprising a targeted-release formulation for delayed release or enteric or colonic release.

6. The composition according to claim 4 for use as a medicament.

7. The composition according to claim 4 for use in the treatment of prevention of Alzheimer's disease.

8. A non-therapeutic method of improving the health status, in particular the gut health status, of a subject in need, the method comprising administering the B. subtilis strain according to claim 1 or a preparation thereof.


SEQUENCE LISTING (5 sequences)

SEQ ID NO: 1 - DNA, 1547 bp, Bacillus subtilis
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 2 - DNA, 1197 bp, Bacillus subtilis
  source 1..1197: mol_type = genomic DNA
gtgaaaaataaatggctgtcttttttttcgggtaaggtccagcttgaattgacgggaagagggattgagcggctccttaatgaatgcacaagacaggggattccggtctttcatgtcaaaaaaaagaaagaagccgtatcgttatatatacagcttcaggatgtacatgcctttcggcgggtgagaagtaaatttaaatgtaaagcccgatttatcaatcggaagggatttcccttcctgttgctgaaatcaaagctgaatatagggtttacgatcggttttgcgatttttttcatccttttgtttttgctgtccaatatggtgtggaaaattgatgtgacaggcgctaagcctgaaacagaacatcaaatgaggcagcatcttaatgaaatcggcgtcaaaaagggccgtctgcagtttttaatgatgtcgcccgaaaaaatacagaaatcattaaccaatggaatagacaatatcacttgggtcggagttgatctgaaggggacgaccattcacatgaaagttgtggagaaaaatgagcccgaaaaagaaaagtatgttagcccgcgcaatattgtcgccaaaaagaaagcaaccattacgagaatgtttgtgcaaaaaggacagcccatggccgccatacacgatcatgttgaaaaggggcagctgcttgtttcgggactgatcggcagcgaagaccatcagcaggaagttgcctcaaaagcagaaatttacggagaaacctggtatagatcagaagtgacagtcccgcttgaaacattatttaacgtctatacgggcaaagtaaggacaaagcacaagctttcttttggttctttggcaatcccgatctgggggatgacgtttaaaaaagaggaattgaagcatccaaaaacagaacaagaaaaacattcgcttcattttctcggatttaagctccctgtatcctatgtcaaagagcaaacgagagaaagtgaagaggctttgcgaaaatatacaaaagaagaagccgttcaagaaggcattaaattgggtaaacaggatgtagaggataaaataggcgaaaacggcgaggtgaaaagtgaaaaagttttgcaccagactgttgagaatggtaaagtaaagttgattattctctaccaagttatagaagatatcgttcaaaccacacctattgtcagggagactgaagaatga

SEQ ID NO: 3 - DNA, 1923 bp, Bacillus subtilis
  source 1..1923: mol_type = genomic DNA
gtggctatggaacagcagcaaaacagttatgatgaaaatcagatacaggtactagaaggattggaagctgttcgtaaaagaccggggatgtatatcggttcgacaaacagcaaaggccttcaccacctggtatgggaaattgtcgacaatagtattgacgaagccctcgccggttattgtacggatatcaatatccaaatcgaaaaagacaacagtatcacggttgtagataatggccgcggtattccagtcggtattcatgaaaaaatgggccgtcctgcggtagaagtcattatgacggtacttcatgccggaggaaaatttgacggaagcggctataaagtatccggaggattacacggtgtaggtgcgtctgtcgtaaacgcactatcaacagagcttgatgtgacggttcaccgtgacggtaaaattcaccgccaaacttataaacgcggagttccggttacagaccttgaaatcattggcgaaacggatcatacaggaacgacgacacattttgtcccggaccctgaaattttctcagaaacaaccgagtatgattatgatctgcttgccaaccgcgtacgtgaattagcctttttaacaaagggcgtaaacatcacgattgaggataaacgtgaaggacaagagcgcaaaaatgaataccattacgaaggcggaattaaaagttatgtagagtatttaaaccgctctaaagaggttgtccatgaagagccgatttacattgaaggcgaaaaggacggcattacggttgaagtggctttgcaatacaatgacagctacacaagcaacatttactcgtttacaaacaacattaacacgtacgaaggcggtacccatgaagctggcttcaaaacgggcctgactcgtgttatcaacgattacgccagaaaaaaagggcttattaaagaaaatgatccaaacctaagcggagatgacgtaagggaagggctgacagcgattatttcaatcaaacaccctgatccgcagtttgagggccaaacgaaaacaaagctgggcaactcagaagcacggacgatcaccgatacgttattttctacggcgatggaaacatttatgctggaaaatccagatgcagccaaaaaaattgtcgataaaggcttaatggcggcaagagcaagaatggctgcgaaaaaagcccgtgaactaacacgtcgtaagagtgctttggaaatttcaaacctgcccggtaagttagcggactgctcttcaaaagatccgagcatctccgagttatatatcgtagagggtgactctgccggaggatctgctaaacaaggacgcgacagacatttccaagccattttgccgcttagaggtaaaatcctaaacgttgaaaaggccagactggataaaatcctttctaacaacgaagttcgctctatgatcacagcgctcggcacaggtattggggaagacttcaaccttgagaaagcccgttaccacaaagttgtcattatgacagatgccgatgttgacggcgcgcacatcagaacacttctgttaacgttcttttacagatatatgcgccaaattatcgaaaatggctacgtgtacattgcgcagccgccgctctacaaggttcaacaggggaaacgcgttgaatatgcgtacaatgacaaggagcttgaagagctgttaaaaactcttcctcaaacgcctaagcctggactgcagcgttacaaaggtcttggtgaaatgaatgccacccagctatgggagacaaccatggatcctagttccagaacacttcttcaggtaactcttgaagatgcaatggatgcggacgagacttttgaaatgcttatgggcgacaaggtagaaccgcgccgaaacttcatagaagcgaatgcgagatacgttaaaaatcttgacatctaa

SEQ ID NO: 4 - DNA, 3582 bp, Bacillus subtilis
  source 1..3582: mol_type = genomic DNA
ttgacaggtcaactagttcagtatggacgacaccgccagcgcagaagctatgctcgcattagcgaagtgttagaattaccaaatctcattgaaattcaaacctcttcttatcagtggtttcttgatgagggtcttagagagatgtttcaagacatatcaccaattgaggatttcactggtaacctctctcttgagttcattgattatagtttaggtgagcctaaatatcctgtagaggaatcaaaagaacgtgatgtgacttactcagctccgctaagagtgaaggttcgtttaattaacaaagaaactggagaggtaaaagaccaagatgtcttcatgggtgatttccctattatgacagatacaggtacttttatcattaacggtgcggaacgtgttatcgtttcccagcttgttcggtctccaagtgtatatttcagtggtaaagtagacaaaaacggtaaaaaaggttttaccgcaactgtcattccaaaccgtggcgcatggttagaatacgaaactgatgcgaaagatgttgtttatgtccgcattgatcgcacacgtaagttgccggttacggttcttttgcgtgctctcggcttcggctccgatcaagagattcttgatctcataggagaaaacgaatacctgcgaaatacgcttgataaagataacacagaaaacagcgacaaagcgttgctggaaatttacgagcgtctccgtcctggagagccgcctacagtagaaaatgcgaaaagcttgcttgattctcgtttctttgatccgaaacgatacgatcttgccaatgtaggacgctataaaattaataaaaaacttcatattaagaatcgcctcttcaatcagagacttgctgaaacgcttgttgatcctgaaacaggagaaatccttgctgaaaaaggccagattcttgatagaagaacgcttgataaagtactgccatacttagaaaacggaatcggttttagaaagctgtatccgaatggcggcgttgttgaagatgaagtgactcttcaatcaattaaaatctttgctccgactgatcaagaaggagaacaggttatcaatgtaatcggcaatgcttacatcgaagaagagattaaaaacatcacgcctgctgatattatttcttcaatcagctacttcttcaacctgctgcacggagtaggcgacacagatgatatcgatcatcttggaaaccgccgtttacgttctgtaggcgagcttctccagaaccaattccgtatcggtttaagccgtatggagcgtgtggttcgtgagagaatgtcaattcaagatacgaatacaattacgcctcagcagctgatcaatattcgtcctgttattgcgtccattaaagagttctttggaagctcacagctttctcaattcatggatcagacgaacccgcttgctgaattaacgcacaagcgtcgtctgtcagcattaggaccgggcggattgacacgtgagcgtgccggaatggaagtgcgtgacgttcactactcccactatggccgtatgtgtccgattgaaacgcctgagggcccgaacatcggtttgatcaactcactatcatcttatgcaaaagtaaaccgttttggctttattgaaacgccatatcgccgcgttgaccctgaaacagggaaggtaacgggcagaatcgattacttaactgctgatgaagaggataactatgttgtcgctcaagcgaatgctcgtcttgatgacgaaggcgcctttattgatgacagcatcgtagctcgtttccgcggggagaacaccgttgtttccagaaatcgtgtagactacatggatgtatcgcctaagcaggttgtatctgctgcgacagcatgtatcccgttcttagaaaacgatgactccaaccgtgccctcatgggagcgaacatgcagcgtcaggctgtgcctttgatgcagccggaagcgccatttgttggaactggtatggaatacgtatcaggaaaagactctggtgccgctgttatttgtaaacaccctggtatcgttgaacgcgtagaagccaaaaacgtttgggttcgccgttatgaagaagtagacggtcaaaaagtaaaaggaaacctggataaatacagcctgctgaaatttgtccgctctaaccaaggtacgtgctacaaccagcgtccgatcgtaagtgtcggcgatgaagtggtaaaaggagaaatccttgctgacggtccttctatggagcttggtgaacttgcacttggccgtaacgtaatggtcggcttcatgacgtgggatggctacaactatgaggatgccatcatcatgagtgaacgcctagtgaaggatgatgtttatacatctatccacattgaagaatacgaatcagaagcacgtgatacgaaacttggacctgaagaaatcactcgcgatattccaaacgtcggtgaagatgcgcttcgcaatcttgatgaccgcggaatcatccgtattggggcagaagtaaaagacggagatcttcttgttggtaaagtaacgcctaaaggcgtaactgaactgactgccgaagaacgccttcttcacgccatctttggcgagaaagcccgcgaggttcgtgatacttctcttcgtgtgcctcatggcggcggcggaattatccatgacgttaaagtcttcaaccgtgaagacggagacgaacttcctccaggtgttaaccagttagtacgcgtatatatcgttcagaaacgtaagatttctgaaggggataaaatggccggtcgtcacggtaacaaaggtgttatctctaagattcttcctgaagaggatatgccttaccttcctgacggcacaccaattgatatcatgcttaacccgctgggcgtaccatcacgtatgaacatcgggcaggtattggaacttcacatgggtatggccgctcgttaccttggcattcacattgcatctcctgtatttgacggagcgcgagaagaggatgtctgggaaacacttgaagaagccggcatgtctcgtgacgccaaaacagtgctttacgacggacgtactggagagccgtttgataaccgtgtatctgtcggtatcatgtacatgatcaaactggctcacatggttgacgataaacttcatgcacgctctacaggcccttactcacttgttacgcagcagcctcttggcggtaaagcgcaatttggcggacagcgttttggtgagatggaggtttgggcacttgaagcttacggtgcggcttacactcttcaagaaattctgactgttaagtctgatgacgtggttggacgtgtgaaaacatacgaagccatcgttaaaggcgacaatgttcctgaaccaggtgttccggaatcattcaaagtattaatcaaagaacttcaaagcttaggtatggatgtcaaaatcctttctggtgatgaagaagaaatagaaatgagagatttagaagacgaagaagatgcgaaacaagctgacggcctggcattatcaggtgatgaagagccggaagaaacagcatctgcagacgttgaacgcgatgtagtaacaaaagaataa

SEQ ID NO: 5 - DNA, 1635 bp, Bacillus subtilis
  source 1..1635: mol_type = genomic DNA
atggcaaaagaaattaagtttagtgaagaagctcgccgcgcaatgcttcgcggtgtcgatgcacttgctgatgctgttaaagtaactttaggaccaaaaggacgcaacgtggttctagagaaaaaattcggttctccgttaatcacaaatgacggtgtaacaatcgctaaagaaatcgagctagaagacgcgtttgaaaacatgggtgctaagcttgttgctgaagtagccagcaaaacaaacgacgttgccggtgacggtacaacaactgcaacagttcttgcgcaagcaatgatccgtgaaggccttaaaaacgtaacagcaggcgctaaccctgtaggcgtgcgtaaagggatggaacaagctgtagcggttgcgatcgaaaacttaaaagaaatttctaagccaatcgaaggcaaagagtctatcgctcaggttgctgcgatctctgctgctgatgaggaagtcggaagccttatcgctgaagcaatggagcgcgtaggaaacgacggcgttatcacaatcgaagagtctaaaggcttcacaactgagcttgaagttgttgaaggtatgcaattcgaccgcggatatgcgtctccttacatggtaactgactctgataagatggaagcggttcttgacaatccttacatcttaatcacagacaaaaaaatcacaaacattcaagaaatccttcctgtgcttgagcaggttgttcagcaaggcaaaccattgcttctgatcgctgaggatgttgaaggcgaagcacttgcaacacttgttgtgaacaaacttcgcggcacattcaacgcagttgctgttaaagctcctggcttcggtgaccgccgtaaagcaatgcttgaagacatcgctgtccttactggcggagaagtcatcacagaagatcttggccttgacctgaaatctactcaaatcgctcaattgggacgcgcttctaaagttgtcgttactaaagaaaacacaacaatcgttgaaggcgctggcgaaacagacaaaatttctgcccgcgtgactcaaatccgcgctcaagtggaagaaacaacttctgaattcgacagagaaaaattacaagagcgtcttgcgaaacttgctggcggcgtagctgtcatcaaagtcggcgctgcgactgaaactgagctgaaagagcgtaaacttcgcatcgaagacgccttgaactcaactcgcgcagctgttgaagaaggcatcgtatccggtggcggtacagcgcttgtaaacgtatataacaaagtcgctgcagttgaagctgaaggcgatgctcaaacaggtatcaacatcgtgcttcgcgcgcttgaagagccaatccgtcaaatcgcacacaatgctggtcttgaaggatctgtcatcgttgagcgcctcaaaaacgaagaaatcggcgtaggcttcaacgctgcaactggcgaatgggtaaacatgatcgaaaaaggtatcgttgacccaactaaagttacacgctcagctcttcaaaacgctgcgtctgtagctgcaatgttcttaacaacagaagctgttgtcgctgacaagccagaagaaaacggtggcggcgcaggaatgcctgatatgggcggcatgggcggtatgggtggaatgatgtaa